(19) United States
(12) Patent Application Publication
(10) Pub. No.: US 2006/0166934 A1
(43) Pub. Date: Jul. 27, 2006

(54) Composition comprising phytosphingosine derivatives for apoptosis induction

(75) Inventors: Tae-Yoon Kim, Seoul (KR); Hye-Jung Kim, Seoul (KR); Shin-Hee Kim, Seoul (KR); Ha-Young Hwang, Seoul (KR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Tae-Yoon Kim, Seoul (KR)

(21) Appl. No.: 10/524,562

(22) PCT Filed: Oct. 8, 2002
(86) PCT No.: PCT/KR02/01875

§ 371 Date: Feb. 14, 2005

(30) Foreign Application Priority Data
Aug. 14, 2002 (KR) 2002/48277
Sep. 11, 2002 (KR) 2002/54936

(51) Int. Cl. A61K 31/685; A61K 31/59

(52) U.S. Cl. 514/78; 514/167

(57) ABSTRACT

The present invention is related to compositions for the induction of apoptosis containing phytosphingosine derivatives as effective components. The present invention is related to compositions for the induction of apoptosis containing Vit D₃ or calcipotriol as an effective component in addition to phytosphingosine derivatives. The compositions of the present invention include pharmaceutical compositions or cosmetic compositions having an activity to induce apoptosis. The present invention offers a method of prevention or treatment of various skin diseases, various tumors, various cancers, etc. that may be prevented or cured by the induction of the activity of apoptosis in living bodies, which is comprised of the steps of administration of compositions for the induction of apoptosis containing phytosphingosine derivatives as effective components and irradiation of UVB to psoriatic lesions. Therefore, the compositions of the present invention are useful for the prevention or treatment of various skin diseases, various tumors, various cancers, etc. that may be prevented or cured by the induction of the activity of apoptosis in living bodies.

### BACKGROUND OF THE INVENTION

1. Field of the Invention

The present invention is related to compositions containing phytosphingosine derivatives for the induction of apoptosis.

2. Description of the Prior Art

Apoptosis refers to a programmed cell death, which is one mode of cell death occurring under the physiological and pathological conditions.

It has been known that while the number of cells is maintained constantly in normal tissues since the proliferation of cells and apoptosis are balanced, cancer cells are proliferated in tumorous tissues since the number of cells is increased due to inadequate apoptosis compared to a rapid proliferation of cells (Raff, M. C., Nature, 356:397, 1992). The factors related to the induction of apoptosis are known to include p53, bcl-2, bcl-XL, caspase, etc. (Wyllie, A., Nature, 389:237, 1997). Once the apoptotic program is activated, programmed cell death starts with blebbing of the membrane, followed by degradation of the chromosomal DNA by nucleases, resulting in condensation and fragmentation of DNA Apoptosis plays an important role in the generation of fetuses and functions of the skin, internal organs, and immunologic organs.

Inflammatory skin diseases are caused by the interaction of many immune cells including lymphocytes with keratinocytes occupying most of skin cells, as immune cells are penetrated into the skin ultimately. These keratinocytes affect on the proliferation of immune cells by secreting many cytokines participating in immune functions, and are supplied with many factors involved in the proliferation of the keratinocytes from immune cells. These cells and surrounding lymph nodes are called skin-associated lymphoid tissues (SALT). In view of this, the skin is regarded to be not only the protective membrane of our bodies simply but also one of immune organs.

Among skin diseases related to disregulation of apoptosis, psoriasis is a disease characterized by hyperproliferation of keratinocytes, and infiltration and activation of various inflammatory cells, in particular, T-cell. Since keratinocytes in psoriatic patients highly proliferate along with angiogenesis, compounds inducing apoptosis in psoriatic patients are suggested to be effective drugs for the managements of psoriasis.

Particularly, it is known that ceramide, a sphingolipid derivative, converted from sphingomyelin by SMase (sphingomyelinase) activated by stimuli such as tumor necrosis factor-α (TNF-α), interleukin-1 (IL-1), interferon-γ (IFN-γ), FAS ligand, and irradiation, acts as a second messenger to mediate cell differentiation, inhibition of cell cycle, proliferation and apoptosis.

Also, it is known that vitamin D₃ or calcipotriol induces apoptosis and cytotoxicity to hyperproliferative keratinocytes.

Further, it has been reported that apoptosis is induced by Smase which is activated by the irradiation of UV light and accelerates formation of ceramide in the cells. Still further, it is known that growth factors and cytokine surface receptors activated by irradiation of UV light stimulate cytokine or growth factor signal transduction pathway.

UV light is divided into UVA (200-290 nm), UVB (290-320 nm), and UVC (320-400 nm) according to the wavelength. It has been reported that these facilitate immune suppression functions and cause apoptosis in vivo or in culture cells.

The inventors of the present invention completed the present invention as they found that the effect of induction of apoptosis was shown significantly in a specific phytosphingosine derivative while they searched for effective compounds for various diseases showing the preventive or treatment effects through induction of the apoptosis.

Proposed in the present invention are compositions for inducing apoptosis having phytosphingosine derivatives as effective components.

### SUMMARY OF THE INVENTION

In the present invention, compositions inducing apoptosis containing phytosphingosine derivatives as effective components are provided for.

In addition to phytosphingosine derivatives, in the present invention, a composition inducing apoptosis containing vitamin D₃ or calcipotriol as an effective component is suggested.

The compositions of the present invention are characterized by that the phytosphingosine derivative is one or more compounds selected from a group comprised of phytosphingosine (PS), phytosphingosine-HCl (PS-HCl), C6-phytosphingosine (C6-PS), CLA-phytosphingosine (CLA-PS), tetraacetyl phytosphingosine (TAPS), and N-acetyl phytosphingosine (NAPS).

The compositions of the present invention include pharmaceutical or cosmetic compositions having the activity to induce apoptosis.

Proposed in the present invention is a method of prevention or treatment of various skin diseases, tumors, cancers, etc. that may be prevented or treated by inducing apoptosis in vivo comprised of the steps of administration of a composition for inducing apoptosis containing phytosphingosine derivatives as effective components and of irradiating UVB to psoriatic lesions.

The above preventive or treatment method is characterized by that a phytosphingosine derivative is one or more compounds selected from a group comprised of phytosphingosine, phytosphingosine-HCl, C6-phytosphingosine, CLA-phytosphingosine, tetraacetyl phytosphingosine, and N-acetyl phytosphingosine.

The compositions of the present invention show the cytotoxic effect in all of human keratinocyte cell line, human skin cancer cell line, human umbilical vein endothelial cells and peripheral blood mononuclear cells.

Among the compositions of the present invention, those having TAPS and NAPS as effective components show a significant apoptosis effect in the human keratinocyte cell line.

Among the compositions of the present invention, those containing TAPS, NAPS and PS as effective components have an effect of suppressing the activity of Th1 cells that are related to inducing of psoriasis, particularly.

Among the compositions of the present invention, those containing vitamin D₃ or calcipotriol, that is used for an effective treatment agent of psoriasis, as an additional effective component, show a significantly increased apoptosis effect compared to other compositions of the present invention containing a phytosphingosine derivative singly.

If the compositions of the present invention are administered and UVB is irradiated, they show a significantly increased apoptosis effect than when a phytosphingosine derivative is administered singly.

The dosage of irradiation of UVB in the method of treatment of psoriasis of the present invention when the compositions of the present invention are administered is 50 mJ/cm²-2 J/cm².

The proteins involved in inducing apoptosis by the compositions of the present invention are caspase-3, p53, Chk1, etc.

Caspase-3 is cleavaged to the active form the maximum 3 hours after the composition of the present invention is treated and shows a remarkable ability to induce apoptosis, etc.

The compositions of the present invention is useful for the prevention or treatment of various skin diseases, tumors, cancers, etc. that may be prevented or treated by inducing apoptosis in vivo.

Concretely, the diseases that may be prevented or cured by the compositions of the present invention include abnormal skin diseases such as eczema, psoriasis, ichthyosis, etc.; skin diseases such as atopic dermatitis, dermatitis, itching, microbism, acne, wound, etc.; abnormal skin diseases induced by a long-time exposure to UV light and dermal aging; skin cancer, etc.

Particularly, the compositions of the present invention are useful for the prevention and treatment of keratinization diseases such as psoriasis, ichthyosis, etc.; abnormal skin diseases induced by a long-time exposure to UV light and dermal aging; and skin cancer.

The compositions of the present invention may additionally contain one or more effective components showing the same or similar functions.

Further, the compositions of the present invention may additionally contain one or more effective components showing other functions.

Still further, the compositions of the present invention may include one or more carriers that are allowable pharmacologically in addition to effective components described in the above. Saline solution, sterilized water, Ringer's solution, buffered saline solution, dextrose solution, malto dextrine solution, glycerol, ethanol, and a mixture of one or more of the above may be used for pharmacologically allowable carriers, and if necessary, other usual additives such as an anti-oxidant, buffer solution, bacteriostatic, etc. may be added. Also, a diluent, dispersion agent, surfactant, binder, and lubricant may be added and formulated additionally. And further, it may be formulated desirably according to each disease or component by using a proper method in the present field or a method disclosed in Remington's Pharmaceutical Science (updated version), Mack Publishing Company, Easton, Pa.

It is desirable to administer the compositions of the present invention locally, and they may be offered in the form of an ointment, cream, emulsion, plaster, powder, impregnated pad, solution, gel, spray, lotion, or suspension.

A phytosphingosine derivative occupies 0.05-10.0 weight %, preferably 0.1-5.0 weight % of the total weight of the composition of the present invention. And the compositions of the present invention are applied to the psoriatic lesion a few times a day at a dose of about 10-30 ml or 10-30 g.

A cosmetic composition of the present invention is not particularly limited to its type of formulation. That is, it may be in the form of a tender lotion, astringent, nutritious lotion, eye cream, nutritious cream, massage cream, cleansing cream, cleansing foam, cleansing water, powder, essence, pack, emulsion, lotion, ointment, gel, polymeric or lipid vesicle or nanosphere or microsphere, soap or shampoo, etc. And in the cosmetic composition of each formulation, a person skilled in the art may select and blend components other than phytosphingosine derivatives according to the type of formulation, purpose of use, etc. of other cosmetics.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 shows graphs showing the cytotoxic effect of phytosphingosine derivatives which are effective components of the composition of the present invention in a dose dependent manner;

FIG. 2 shows graphs in which the effects of the compositions of the present invention on the cytotoxicity in immune cells separated from spleen of mice are observed;

FIG. 3 shows graphs in which the effects of the compositions of the present invention on the cytotoxicity of peripheral blood mononuclear cells (PBMC) from human are observed;

FIG. 4 shows graphs in which the effects of the compositions of the present invention on the activity of human Th1 cells by the mixed leucocyte reaction are observed;

FIG. 5 shows graphs in which the effects of the compositions of the present invention on the activity of human Th1 cells by the reaction among allogenic cells are observed.

FIG. 6 shows diagrams showing the effect of the compositions of the present invention on the apoptosis by the TUNEL assay;

FIG. 7 shows diagrams showing the effect of NAPS and TAPS which are effective components of the compositions of the present invention on the apoptosis in a time dependent manner at a concentration of 30 μM;

FIG. 8 is a diagram showing the effect of TAPS which is an effective component of the compositions of the present invention on cell cycle using FACS;

FIG. 9 is a diagram in which the effect of TAPS which is an effective component of the compositions of the present invention on the mitosis is observed;

FIG. 10 is a diagram in which genes involved in apoptosis induced by TAPS which is an effective component of the compositions of the present invention is observed;

FIG. 11 is a diagram in which the effect of NAPS and TAPS which is effective components of the compositions of the present invention on the increase of the cleavaged active caspase-3;

FIG. 12 is a diagram showing the expression of p53 and Chk1 proteins at a concentration of 30 μM of TAPS which is an effective component of the compositions of the present invention;

FIG. 13 is a diagram showing the expression of Chk1 protein according to the time of NAPS and TAPS which are effective components of the compositions of the present invention; and

FIG. 14 is a diagram in which the effects of treatment with NAPS, an effective component of the compositions of the present invention, 5-7 days after it is applied to the psoriatic lesion are observed.

### DETAILED DESCRIPTION OF A PREFERRED EMBODIMENT OF THE INVENTION

Hereinafter, a preferred embodiment of the present invention is presented in order to assist understanding of the present invention.

Firstly, the cytotoxicity is measured by the MTT assay. Phytosphingosine (PS), phytosphingosine-HCl (PS-HCl), C6-phytosphingosine (C6-PS), CLA-phytosphingosine (CLA-PS), tetraacetyl phytosphingosine (TAPS), and N-acetyl phytosphingosine (NAPS) are dissolved into DMSO to have the final concentration of 1-100 μM.

1. Effects of the Compositions of the Present Invention on the Cytotoxicity in the Human Keratinocyte Cell Line

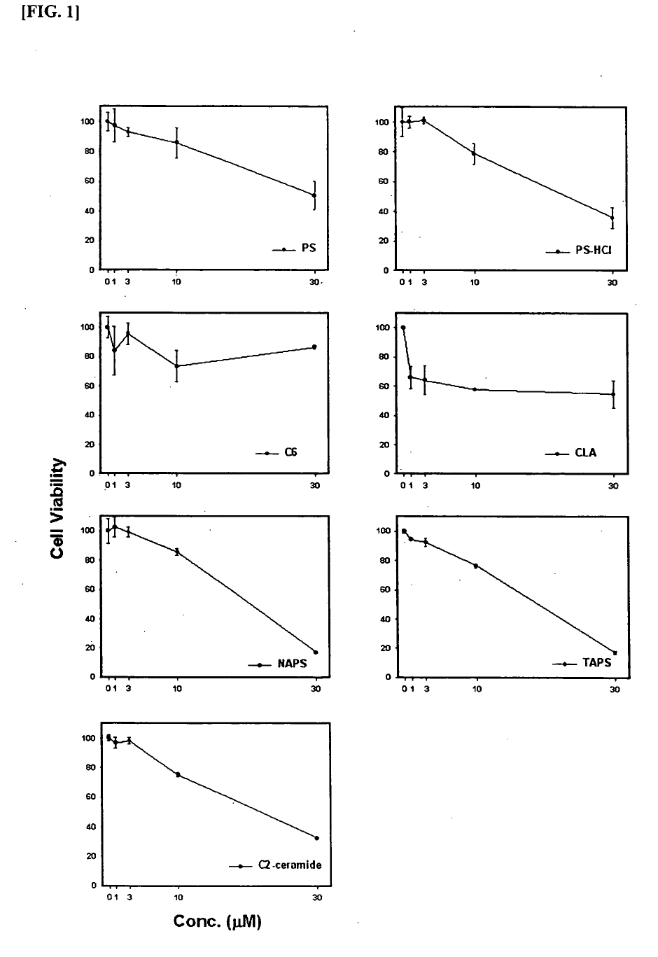

In order to study the effects of the compositions of the present invention on the cytotoxicity in the human keratinocyte cell line HaCaT cells, the cell viability is analyzed by the MTT (3-[4,5-dimethylthiazol-2-yl]-2,5-diphenyltetrazolium bromide) assay. HaCaT cells are supplied by Professor N. Fuseng of German Cancer Research in Germany. HaCaT cells are seeded in a density of 1×10⁶ cells in a 100-nm dish and cultured in DMEM (Dulbecco's Modified Eagle Medium) containing 10% fetal bovine serum (FBS, GIBCO), 100 units/ml penicillin, and 100 μg/ml streptomycin for 48 hours. They are treated with trypsin and 1-2×10⁴ cells per well are seeded again in a 96-well plate by using a serum-free culture medium, about 3 hours after which they are cultured by treatment with phytosphingosine derivatives. After they are cultured for 24 hours, the MTT reagent is added at a concentration of 2 mg/ml, cultured for 4 hours, suspended in DMSO after removing the culture medium completely in order to measure O.D. at 540 nm. C2-ceramide is used for comparison. The results of measurement are shown in Table 1 and FIG. 1 as follows:

TABLE 1
Cell viability of phytosphingosine derivatives versus concentration (1-30 μM)

| | PS | PS-HCl | C6-PS | CLA-PS | NAPS | TAPS | C2-ceramide |
|---|---|---|---|---|---|---|---|
| con | 100 ± 6.2 | 100 ± 9.6 | 100 ± 7.3 | 100 ± 0.6 | 100 ± 8.6 | 100 ± 1.2 | 100 ± 2.0 |
| 1 μM | 97 ± 11.1 | 100 ± 4.1 | 84 ± 16.8 | 66 ± 7.8 | 103 ± 6.9 | 94 ± 0.1 | 97 ± 3.8 |
| 3 μM | 93 ± 3 | 101 ± 2.2 | 96 ± 7.2 | 64 ± 9.9 | 99 ± 3.5 | 93 ± 2.9 | 98 ± 2.5 |
| 10 μM | 86 ± 10.1 | 79 ± 7.0 | 74 ± 10.1 | 58 ± 0.4 | 86 ± 2.1 | 76 ± 1.4 | 75 ± 1.3 |
| 30 μM | 51 ± 9.6 | 36 ± 6.8 | 87 ± 1.3 | 55 ± 9.1 | 17 ± 0.8 | 17 ± 1.1 | 33 ± 0.6 |

As shown in Table 1 and FIG. 1, all of 6 kinds of phytosphingosine derivatives of the present invention suppresses proliferation of cells. It is seen particularly that NAPS and TAPS have a more superior cytotoxic effect than C2-ceramide has by 16% as C2-ceramide shows a 67% cytotoxicity while NAPS and TAPS show a 83% cytotoxicity at 30 μM.

2. Effects of the Compositions of the Present Invention on the Cytotoxicity in the Human Skin Cancer Cell Line (A431)

In order to study the effects of the compositions of the present invention on the cytotoxicity in A431 cells, human skin cancer cell line, the MTT assay is used. The method of cell culture and MTT assay are the same as those in the above method. C2-ceramide is used for comparison. The results of measurement are shown in Table 2 below:

TABLE 2
Cell viability of phytosphingosine derivatives versus concentration (3-50 μM)

| | PS | PS-HCl | C6-PS | CLA-PS | NAPS | TAPS | C2-ceramide |
|---|---|---|---|---|---|---|---|
| con | 100 ± 10.8 | 100 ± 10.8 | 100 ± 10.8 | 100 ± 10.8 | 100 ± 10.8 | 100 ± 10.8 | 100 ± 10.8 |
| 3 μM | 85 ± 10.7 | 105 ± 5.8 | 106 ± 9.7 | 91 ± 11.0 | 100 ± 10.2 | 113 ± 11.2 | 113 ± 19.8 |
| 10 μM | 30 ± 1.9 | 109 ± 8.6 | 61 ± 8.1 | 98 ± 12.9 | 75 ± 6.9 | 83 ± 13.9 | 97 ± 14.7 |
| 30 μM | 2 ± 0.1 | 7 ± 1.6 | 57 ± 10.4 | 60 ± 6.2 | 31 ± 10.0 | 12 ± 1.7 | 82 ± 1.4 |
| 50 μM | 2 ± 0.2 | 2 ± 0.2 | 44 ± 9.6 | 65 ± 5.5 | 1 ± 0.2 | 5 ± 1.2 | 74 ± 12.8 |

As shown in Table 2, it is seen that PS, PS-HCl, NAPS, and TAPS have a more superior cytotoxic effect than that of C2-ceramide by 69-72% as C2-ceramide shows a 26% cytotoxicity while PS, PS-HCl, NAPS, and TAPS show a 95-98% cytotoxicity at 50 μM.

3. Effects of the Compositions of the Present Invention on the Cytotoxicity in Human Umbilical Vein Endothelial Cells (HUVEC)

The MTT assay is used for the above purpose. Primary human umbilical vein endothelial cells are cultured from the human umbilical vein. The method of cell culture and the MTT assay are the same as those in the above. C2-ceramide is used for comparison. The results of measurement are shown in Table 3 below:

TABLE 3
Cell viability of phytosphingosine derivatives versus concentration
(3.5-30 μM)

| | PS | NAPS | TAPS | C2-ceramide |
|---|---|---|---|---|
| con | 100 ± 10.6 | 100 ± 8.5 | 100 ± 10.1 | 100 ± 15.7 |
| 3.5 μM | 64 ± 4.7 | 66 ± 7.5 | 47 ± 8.4 | 90 ± 11.6 |
| 7 μM | 19 ± 1.8 | 51 ± 10.5 | 22 ± 1.9 | 60 ± 7.7 |
| 15 μM | 14 ± 0.9 | 19 ± 3.4 | 15 ± 0.7 | 61 ± 8.4 |
| 30 μM | 13 ± 1.0 | 14 ± 0.6 | 9 ± 0.5 | 34 ± 2.3 |

As shown in Table 3, it is seen that PS, NAPS, and TAPS have a more superior cytotoxic effect than that of C2-ceramide by 20-25% as C2-ceramide shows a 66% cytotoxicity while PS, NAPS, and TAPS show a 86-91% cytotoxicity at 30 μM.

4. Effects of the Compositions of the Present Invention on the Cytotoxicity in Immune Cells Separated from Spleen of Mice

The effect of the composition of the present invention on the cytotoxicity in immune cells (T cells, B cells, macrophages, monocytes, etc.) was examined.

The spleen was removed from normal mice (BDF) and single cells were obtained from the spleen by mechanical aggregation method. And mononuclear cells containing T cells, B cells, macrophages and monocytes were isolated from single cells by density gradient centrifugation method using Ficoll Hypaque. Briefly, single cells were treated with PBS and overlayed on Ficoll Hypaque and centrifuged at 1500 rpm for 30 min. After centrifugation, the cells around the boundary of the Ficoll-Hapaque were collected and washed three times with PBS.

The isolated cells were cultured for 24 hours in the presence of 1, 10, 50 and 100 μM of NAPS, TAPS, and PS to examine the effect of cytotoxicity, i.e. the degree of proliferation of the cells.

C2-ceramide was used as control. The medium used in this experiment was RPMI 1640 suplemented with 10% of FBS and 100 μg/ml of penicillin and streptomycin.

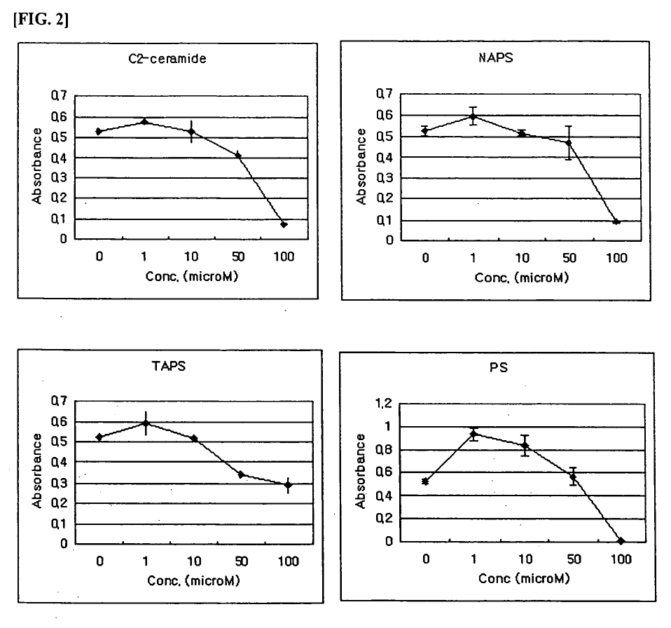

The effect of cytotoxicity was measured by MTT-assay as shown in FIG. 2.

The results in FIG. 2 show that IC₅₀ of C2-ceramide, NAPS, and PS was about 75 μM, and that of TAPS was 100 μM (IC₅₀ means the concentration that has the half effect.). The result shows that the compositions of the present invention have the cytotoxicity to immune cells obtained from spleen of mice.

5. Effects of the Compositions of the Present Invention on the Cytotoxiciy in Peripheral Blood Mononuclear Cells (PBMC) from Human Blood

The effects of the composition of the present invention on the cytotoxicity in immune cells (T cells, B cells, macrophages, monocytes, etc.) obtained from human blood were examined.

The blood was obtained from healthy volunteers and PBMC from the blood by mechanical aggregation method. And mononuclear cells containing T cells, B cells, macrophages and monocytes were isolated from single cells by density gradient centrifugation method using Ficoll Hypaque. Briefly, single cells were treated with PBS and overlayed on Ficoll Hypaque and centrifuged at 1500 rpm for 30 min. After centrifugation, the cells around the boundary of the Ficoll-Hapaque were collected and washed three times with PBS. Each 2×10⁵ PBMC obtained from different volunteers was mixed with each other and cultured for 5 days on the 96-well plate. And RPMI with 10% of serum was used as a medium.

The isolated cells were cultured for 24 hours in the presence of 1, 10, 50 and 100 μM of NAPS, TAPS, and PS to examine the effect of cytotoxicity, i.e. the degree of proliferation of the cells.

C2-ceramide was used as control.

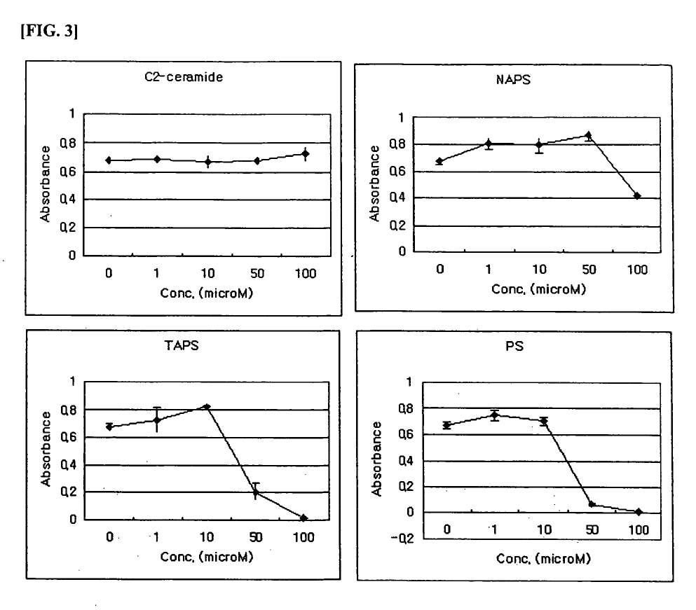

The effect of cytotoxicity was measured by MTT-assay as shown in FIG. 3.

The results in FIG. 3 show that C2-ceramide did not show the cytotoxicity when it was treated up to 100 μM. However, NAPS, TAPS and PS showed the strong cytotoxicity to PBMC. Particularly, 100 μM of NAPS and 50 μM of TAPS shows the strong cytotocicity.

6. Effects of the Compositions of the Present Invention on the Activation of Th1 Cells Associated with the Induction of Psoriasis.

6-1. Mixed Leukocyte Reaction (MLR)

Whether the composition of the present invention has a suppressive effect on the Th1 cells activation which isolate IFN-X, IL-12, etc. by reacting with TNBSO₃ (heptene) was examined.

The mononuclear cells obtained from spleen of mice as described the above 4 were used for responder cells, and TNBSO3-conjugated cells were used for stimulator cells. The stimulator cells were obtained as follows:

The mononuclear cells were adjusted to be 2×10⁷ cells/ml in PBS and the same volume of 20 mM TNBSO3 was added and incubated at 37° C. for 10 min in the dark state. After incubation, the cells wrer washed three times with PBS.

Each 2×10⁵ responder cells and stimulator cells were mixed with each other and cultured for 5 days on the 96-well plate. And RPMI with 10% of serum was used as a medium.

The above cells were cultured for 24 hours in the presence of 1, 10, 50 and 100 μM of NAPS, TAPS, and PS to examine the proliferation of the cells.

C2-ceramide was used as control.

The suppressive effect on Th1 cells activation was measured by MTT-assay as shown in FIG. 4.

The results in FIG. 4 show that C2-ceramide, NAPS and PS showed a suppressive effect on Th1 cells activation at 50 μM, and TAPS at 10 μM. Accordingly, the results showed that all of the compositions of the present invention had a suppressive effect on Th1 cells activation by TNBSO₃.

6-2. Reactions Among Allogenic Cells

Whether the composition of the present invention has a suppressive effect on the Th1 cells activation by allogenic cells in immune reation was examined.

PBMC obtained from different volunteers as described in the above 5 were used for responder cells and stimulator cells respectively. PBMC for stimulator cells were used after X-ray irradiation (3000 rad) to inhibit the proliferation of stimulator cells.

Each 2×10⁵ responder cells and stimulator cells were mixed with each other and cultured for 5 days on the 96-well plate. And RPMI with 10% of serum was used as a medium.

The above cells were cultured for 24 hours in the presence of 1, 10, 50 and 100 μM of NAPS, TAPS, and PS to examine the proliferation of the cells.

C2-ceramide was used as control.

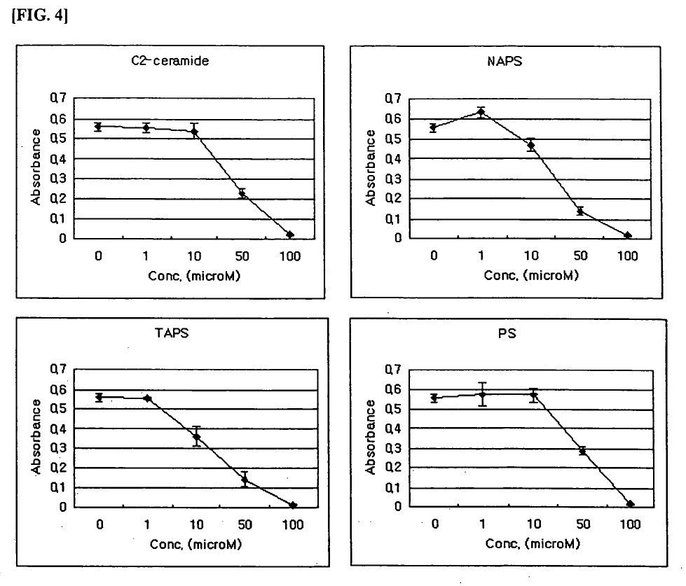

The suppressive effect on Th1 cells activation was measured by MTT-assay as shown in FIG. 5.

The results in FIG. 5 show that while C2-ceramide showed almost no suppressive effect, NAPS, TAPS and PS showed a suppressive effect on Th1 cells activation against allogenic cells at 50 μM, in particular, TAPS and PS suppress completely

Induction of apoptosis of HaCaT cells by the compositions of the present invention is observed in the TUNEL-TdT-mediated dUTP nick end labeling-assay by using in situ cell death detection kit, POD (Enzo, 1684817, Boeringer Mannhein).

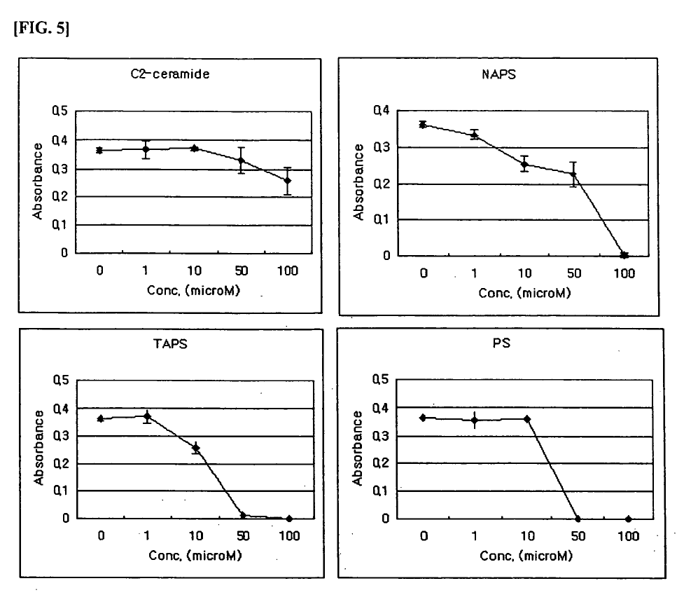

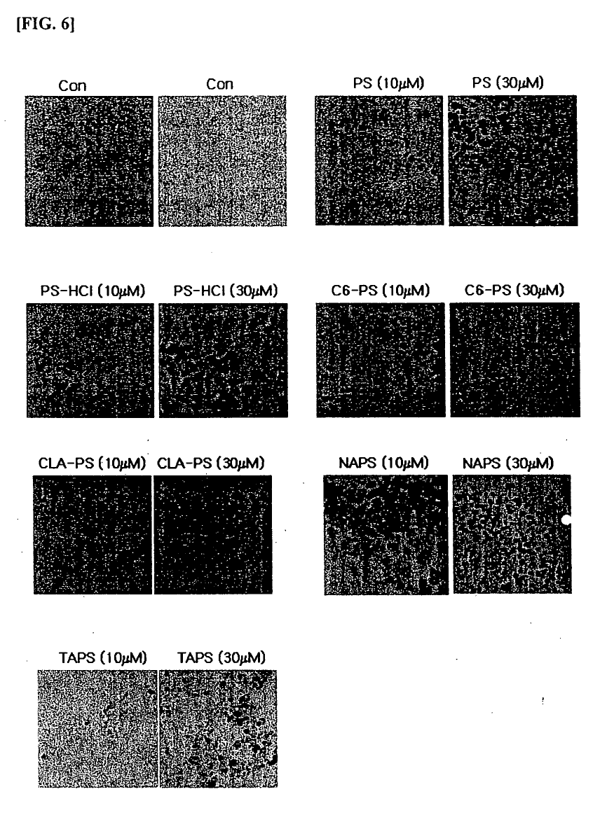

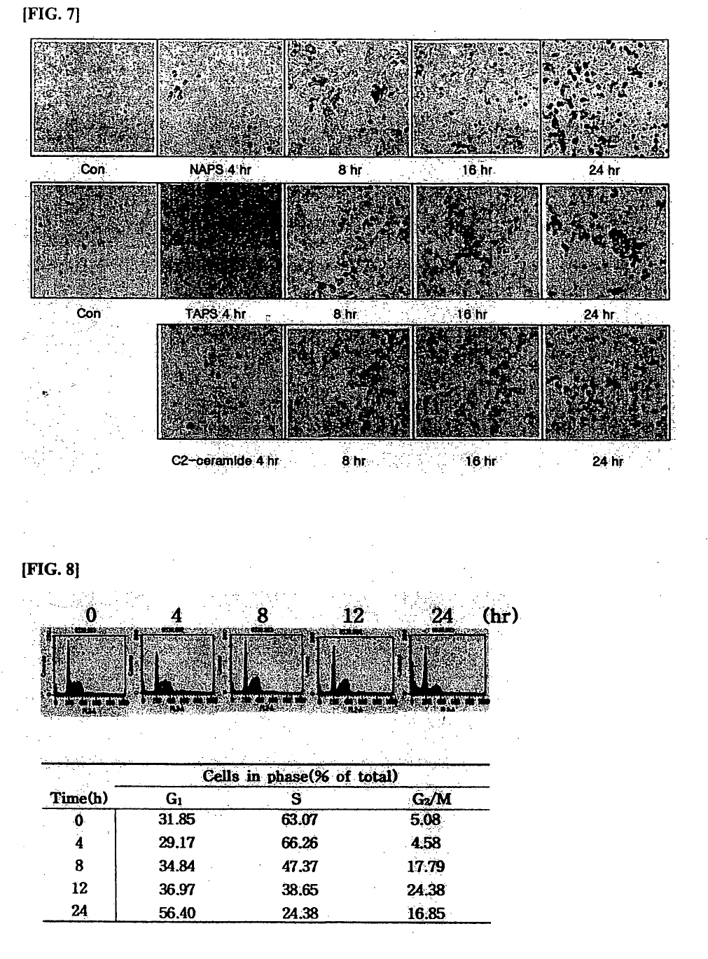

In the TC chamber (Lab-TEK chamber Slide w/cover Permanox Slide sterile 1 well, 177410), 1×10⁶ HaCaT cells are seeded and cultured on the DMEM medium for longer than 18 hours and treated with phytosphingosine derivatives. The cells are fixed 24 hours after treating with drugs, and the endogenous peroxidase is blocked with a blocking solution (3% H₂O₂ in MeOH), and permeation of cells is increased by using a permeabilization solution (0.1% Triton X-100 in 0.1% sodium citrate). Then apoptotic cells are labeled by using the mixture of tunnel reaction and are colored by using the DAB substrate (DAKO, K3465). Colored cells are observed by using a microscope. The results of measurement are shown in FIGS. 6 and 7.

FIG. 6 are diagrams showing the results of the tunnel assay performed by treating 10 μM and 30 μM phytosphingosine derivatives for 24 hours. All of phytosphingosine derivatives cause cell death, and particularly, NAPS and TAPS show the most significant effect of cell death.

FIG. 7 is a diagram showing the effect of inducing apoptosis of NAPS and TAPS at a concentration of 30 μM in a time dependent manner. It is seen that apoptosis is induced 4 hours after treating NAPS and TAPS to have a concentration of 30 μM.

The effects of the compositions of the present invention on the cell cycle are reviewed.

In the flow cytometric analysis, the cells are treated with trypsin according to each time interval, collected and washed with PBS. They are fixed by using 80% ethanol, and propidium iodide and RNase are added to them. Thereafter, the cell cycle is analyzed by using a flow cytometer. The results are shown in FIG. 8.

As shown in FIG. 8, the trend is shown to be that S phase is reduced up to 12 hours after treating with TAPS and G₂/M phase is increased relatively. Thereafter, the trend is shown to be that G₂/M phase is reduced continuously up to 24 hours and sub G₁ phase is increased relatively. Sub G₁ phase refers to apoptotic cells, and it is seen that induction of apoptosis by phytosphingosine derivatives is increased rapidly after 24 hours. It may be viewed that such increase in the number of apoptotic cells comes from the cells in G₂/M phase of which distribution is reduced at the same time interval (The increase in the number of cells in G₁ phase after 24 hours is the result of analysis of living cells except for apoptotic cells.).

Next, the cell cycle is observed according to the immunofluorescence method. In order to look into from which step among G₂/M phase the induction of apoptosis by TAPS is induced, mitosis is observed by using β-tubulin.

HaCaT cells are cultured on a dish with a cover slip and treated with TAPS (30 μM). The cells are washed with PBS at each time interval, fixed with 5% paraformaldehyde for 15 minutes, and reacted with β-tubulin antibody (1:100) for 1 hour. They were washed with PBS and reacted with Cy3-conjugated anti-mouse IgG secondarily. For DNA staining, 0.3 μg/ml of DAPI (Sigma) is added to the cells, which are observed by using a fluorescent microscope. The results are shown in FIG. 9.

As shown in FIG. 9, more cells with chromosomes condensed are observed 24 hours after treating with TAPS than 12 hours after. Therefore, it is seen that the mechanism of apoptosis occurs during the metaphase of mitosis.

In order to study the synergistic effect in the cytotoxicity when the compositions of the present invention are administered in combination with Vit D₃ or calcipotriol, the cell viability is analyzed by MTT assay method in HaCaT cells human keratinocyte cell line.

To HaCaT cells, 1 μM of Vit D₃ or calcipotriol, or 10 μM each of NAPS and TAPS, is administered singly or in combination with each other, and they are cultured for 24 hours. And the effects of these drugs on the cytotoxicity according to the single or combined administration are observed by MTT assay. C2-ceramide is used for comparison. The results are shown in Table 4.

TABLE 4
Cell viability according to combined administration of the compositions
of the present invention with Vit D₃ or calcipotriol

| | | NAPS | TAPS | C2-ceramide |
|---|---|---|---|---|
| | control | (10 μM) | (10 μM) | (10 μM) |
| Control | 100 ± 5 | 86 ± 2 | 76 ± 2 | 78 ± 2 |
| Vit D₃ (1 μM) | 81 ± 3 | 34 ± 4 | 34 ± 4 | 71 ± 9 |
| calcipotriol | 77 ± 5 | 24 ± 3 | 36 ± 8 | 70 ± 4 |
| (1 μM) | | | | |

As shown in Table 4, the administration of 1 μM Vit D₃ or calcipotriol for 24 hours shows the toxicity of 19% or 23%, respectively. While single administration of 10 μM NAPS shows the cytotoxicity of 14% and of 10 μM TAPS does that of 24%, combined application with Vit D₃ or calcipotriol shows the cytotoxicity of greater than 66% or 76%, respectively, in case of NAPS, and 66% or 64%, respectively, in case of TAPS. It is observed that the cytotoxic effect is increased significantly by simultaneous administration of Vit D₃ and calcipotriol or NAPS and TAPS rather than by their single administration.

In order to study the synergistic effect in the cytotoxicity by the cotreatment with administration of the compositions of the present invention and with UVB irradiation, the cell viability is analyzed by MTT assay method in HaCaT cells.

On a 100-mm dish, 1×10⁶ HaCaT cells are seeded and cultured in DMEM containing 10% fetal bovine serum, 100 units/ml penicillin, and 100 μg/ml streptomycin for 48 hours. They are treated with trypsin, and 1-2×10⁴ cells per well are seeded on a 96-well plate again by using a serum-free medium, which is removed after about 3 hours. HaCaT cells are washed with PBS three times, and 3.5 ml of PBS per plate is added. It is then irradiated with 200 J/m² of UVB. After irradiation, the medium is replaced with a DMEM medium. Then it is treated with the phytosphingosine derivatives of the present invention, and cultured for 24 hours. After 2 mg/ml of the MTT reagent is added, it is cultured for 4 hours. The medium is removed completely, the cells are suspended in DMSO, and O.D. is measured at 540 nm. The results of measurement are shown in Table 5 as follows:

TABLE 5
Cell viability according to combined processing of administration of the
compositions of the present invention and irradiation of UVB

| | | | | UVB + | | UVB + | | UVB + |
|---|---|---|---|---|---|---|---|---|
| | PS | UVB + PS | PS-HCl | PS-HCl | NAPS | NAPS | TAPS | TAPS |
| Con | 100 ± 2.4 | 100 ± 2.4 | 100 ± 5.2 | 100 ± 5.2 | 100 ± 9.7 | 100 ± 6.4 | 100 ± 8.5 | 100 ± 8.5 |
| UVB | — | 38 ± 3.3 | — | 39 ± 3.5 | — | 30 ± 6.6 | — | 36 ± 8.0 |
| (200 J/m²) | | | | | | | | |
| 1 μM | 133 ± 3.4 | 38 ± 7.9 | 83 ± 7.9 | 39 ± 4.5 | 130 ± 10.0 | 27 ± 5.8 | 111 ± 7.0 | 27 ± 7.1 |
| 3 μM | 93 ± 5.5 | 30 ± 3.3 | 78 ± 10.0 | 24 ± 2.0 | 117 ± 9.7 | 17 ± 1.6 | 97 ± 10.0 | 27 ± 3.9 |
| 5 μM | 85 ± 10.6 | 28 ± 2.3 | 78 ± 6.7 | 25 ± 1.9 | 110 ± 14.0 | 11 ± 1.3 | 83 ± 8.0 | 16 ± 3.2 |
| 10 μM | 71 ± 7.6 | 3 ± 0.9 | 75 ± 8.2 | 2 ± 0.5 | 67 ± 12.0 | 1 ± 0.1 | 43 ± 4.0 | 2 ± 0.1 |

As shown in Table 5, single irradiation of UVB shows the cytotoxicity of about 61-70%, treating with only 10-μM NAPS shows that of 33%, and treating with only 10-μM TAPS shows that of 57%. The cytotoxic effect is shown to be about 98% or greater in case of the combined use of the administration of NAPS or TAPS and irradiation of UVB.

Therefore, in case of the cotreatment of the administration of NAPS or TAPS composition and irradiation of UVB, it is observed that the cytotoxic effect is increased significantly compared to the case of single treatment of them.

In the subsequent study, genes and proteins involved in induction of apoptosis by the compositions of the present invention are confirmed.

Firstly, in order to look into the genes involved in apoptosis induced by the compositions of the present invention, TAPS is treated at the dose of 30 μM for 24 hours, from which mRNA is isolated. Then it is observed by using an apoptosis array kit.

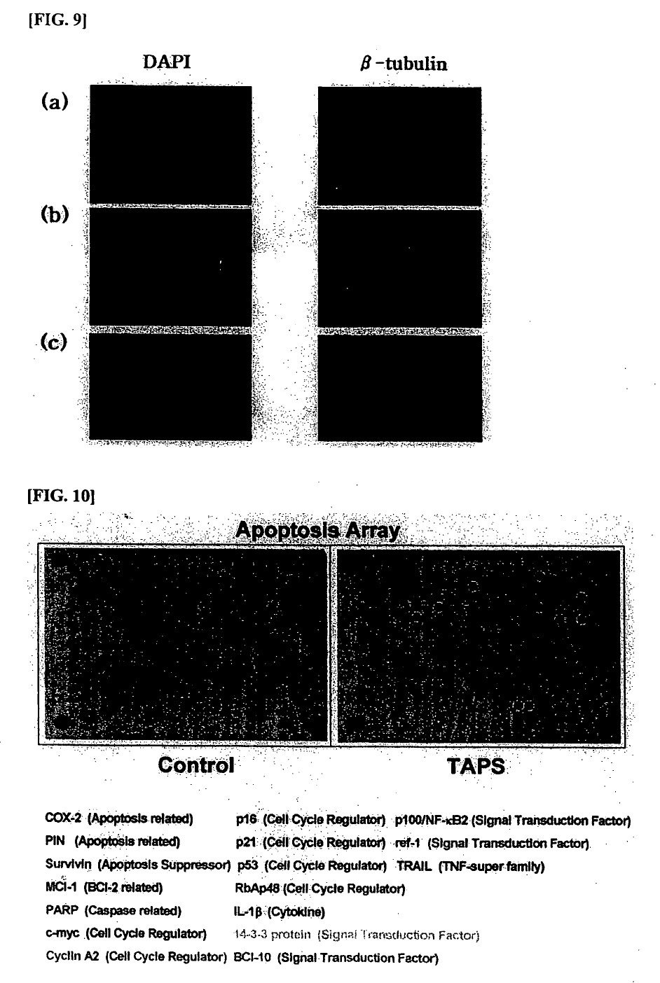

The labeled cDNA is synthesized by using apoptosis-specific primers offered by Human Apoptosis Expression Assay (R&D System, Minneapolis, Minn.), which is used for a probe in incubation with 2 μg of the total RNA at 65° C. overnight. It is then washed with Washing Solution I (0.5×SSPE, 1% (w/v) SDA) three times at a room temperature and with Washing Solution II (0.1% SSPE, 10% (w/v) SDS) for 1 hour at 65° C. The washed membranes are exposed to X-ray films at 70° C., and developed. The results are shown in FIG. 10.

As shown in FIG. 10, as to apoptosis-related genes, the expression of COX-2 and PIN genes is increased, survivin, which is apoptosis suppressor, is reduced, and Mcl-1 and Bcl-10, which are Bcl-2-related genes, are increased. Also, IL-1β among cytokines is increased, p21 which is a cell cycle regulator is increased, but p53 is decreased.

In the confirmation of apoptosis-related proteins, the immunoblot analysis is performed in order to look into the effects of the compositions of the present invention on the expression of caspase-3, p53, and Chk1 proteins related to the induction of apoptosis.

HaCaT cells are cultured on a 100-mm dish and collected at the indicative time. The proteins are extracted by adding 500 μl of RIPA lysis buffer (1% Nonidet P-40, 1% sodium deoxycholate, 0.1% SDS, 0.15 M SDS, 0.01 M sodium phosphate, pH 7.2, 2 mM EDTA, 50 mM sodium fluoride and 0.2 mM sodium vanadate), and the concentration is measured by using the Bradford method. 1

Extracted proteins is electrophoresed on 12% polyacrylamide gel, and they are transferred to the nitrocellulose membrane. The membrane blocked with 5% non-fat milk is reacted with the primary antibodies (caspase-3, p53, and Chk1), washed, and reacted with the secondary HRP-conjugated antibody. The intensity of proteins is analyzed by using the ECL kit. The results are shown in FIGS. 11, 12, and 13.

As shown in FIG. 11, according to the results of observation of the induction of caspase-3 by NAPS, TAPS, and C2-ceramide at the indicative time, the cleavage to the active caspase-3 is increased significantly 30 minutes after NAPS treatment and shows 6 times greater expression of induction to the maximum within 3 hours, and the increased active caspase-3 is returned to the level of the control group within 24 hours after it is maintained for 12 hours.

On the other hand, although TAPS begins to increase active caspase-3 production late compared to NAPS does, 5 folds greater expression of induction to the maximum is shown within 3 hours after the drugs are treated, and its increase is maintained for 24 hours.

Also, C2-ceramide increases production of active caspase-3 to the maximum within 3 hours after the drugs are treated, but the increase is significantly weak compared to NAPS and TAPS (increase by 2.5 folds). Through the above results, it is seen that phytosphingosine derivatives have a superior ability to induce apoptosis, and is more superior than C2-ceramide known conventionally in the efficacy related to the induction of apoptosis.

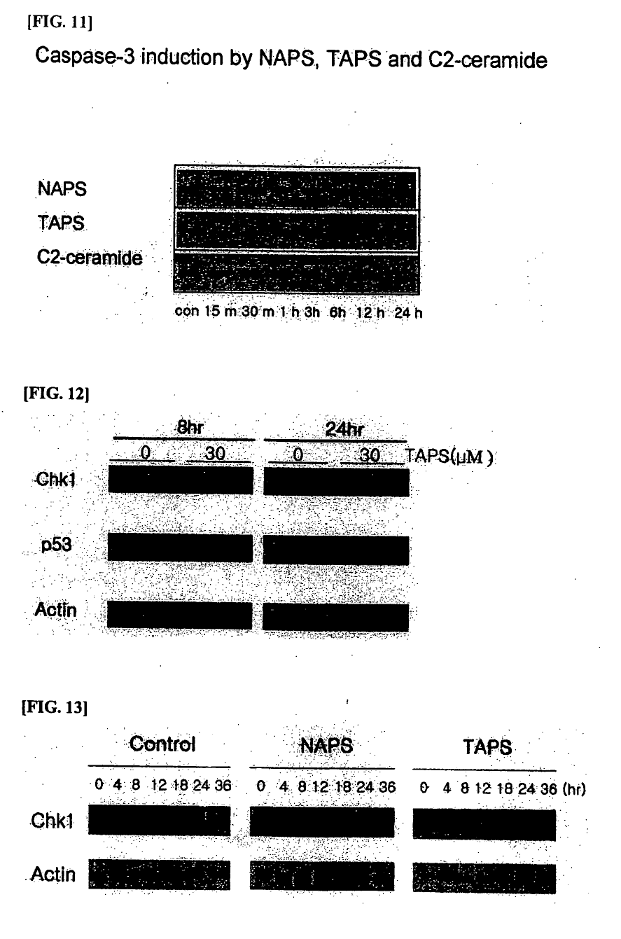

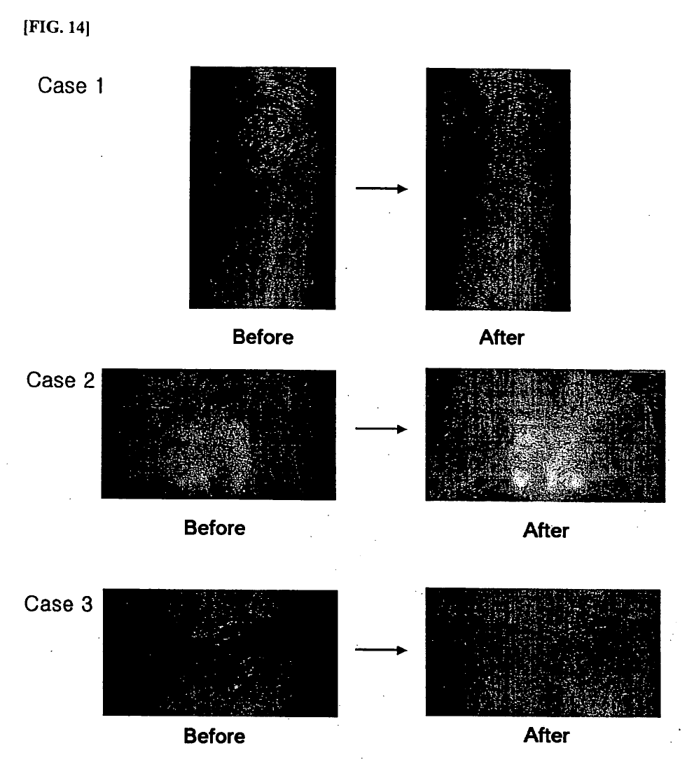

As shown in FIGS. 12 and 13, p53 and Chk1 proteins show the tendency of rapid reduction at 24 hour after treated which apoptosis occurs although they are not changed greatly by treatment with TAPS at 8-hour. These results mean that p53 and Chk1 paths are involved in the induction of apoptosis by ceramide. It is seen, particularly, that Chk1 is a protein involved in the inhibition of G₂/M phase by the damage of DNA.

In the next study, the effects of treatment of psoriasis by the compositions of the present invention are reviewed. NAPS, which is an effective component of the compositions of the present invention, is made to have a concentration of 0.5%, and a cream formulation is manufactured at a composition ratio of the following components and used for a reagent:

| | |
|---|---|
| NAPS | 0.5 g |
| Stearic acid | 1.0 g |
| Cetyl alcohol | 1.4 g |
| Stearyl alcohol | 1.4 g |
| Glyceryl monostearate | 2.0 g |
| Solbitan monostearate | 0.2 g |
| Methyl paraben | 0.2 g |
| Propyl paraben | 0.1 g |
| Mineral oil | 10.0 g |
| Caprylic/capric acid | 3.0 g |
| MDF (meadow foam seed oil) | 3.0 g |
| Dimethicone (methyl polysiloxane) | 0.5 g |
| Solbitan cesquiolate | 0.2 g |
| Twin 60 | 1.2 g |
| Disodium EDTA | 0.02 g |
| Glycerin | 3.0 g |
| Trimethanolamine | 0.2 g |
| Sepigel 305 | 0.5 g |
| Zermol 115 | 0.2 g |
| Purified water | 71.28 g |

Four each 20- to 40-year-old male and female patients are in the experimental group. The reagent manufactured in the above is applied to the psoriatic lesions of 8 patients and the changes are observed. Improved symptoms of each psoriatic lesions are shown in FIG. 14 (Case 1: elbow, Case 2: forehead, Case 3: knee). It is confirmed that 7 among 8 patients have considerably improved symptoms 5-7 days after application of cream preparations of the compositions of the present invention. Therefore, it is seen that the compositions of the present invention are effective for the treatment of psoriasis.

Six kinds of phytosphingosine derivatives used for the present invention show superior effects in the cytotoxicity and induction of apoptosis. Accordingly, the compositions of the present invention containing these phytosphingosine derivatives as effective components are useful for the prevention or treatment of various skin diseases, various tumors, various cancers, etc. that may be prevented or cured by the induction of apoptosis in living bodies.

While the invention has been described in terms of a single preferred embodiment, those skilled in the art will recognize that the invention can be practiced with modification within the spirit and scope of the appended claims.

1. A pharmaceutical composition for the induction of apoptosis containing phytosphingosine derivatives as effective components.

2. The pharmaceutical composition for the induction of apoptosis of claim 1, wherein said phytosphingosine derivatives are one or more compounds selected from a group comprised of phytosphingosine, phytosphingosine-HCl, C6-phytosphingosine, CLA-phytosphingosine, tetraacetyl phytosphingosine, and N-acetyl phytosphingosine.

3. The pharmaceutical composition for the induction of apoptosis of claim 1, wherein Vitamin D₃ or calcipotriol is contained additionally in said phytosphingosine derivatives.

4. The pharmaceutical composition of claim 1, which is useful for the prevention or treatment of various skin diseases, various tumors, various cancers, and other diseases that may be prevented or cured by the induction of the activity of apoptosis in living bodies.

5. The pharmaceutical composition for the induction of apoptosis of claim 4, which is useful for the prevention or treatment of abnormal skin diseases such as eczema, psoriasis, ichthyosis, and others; skin diseases such as atopic dermatitis, dermatitis, itching, microbism, acne, wound, and others; abnormal skin diseases induced by a long-time exposure to UV light and dermal aging; and skin cancer and others.

6. The pharmaceutical composition for the induction of apoptosis of claim 5, which is useful for the prevention or treatment of abnormal skin diseases such as psoriasis, ichthyosis, and others and abnormal skin diseases induced by a long-time exposure to UV light and dermal aging.

7. The pharmaceutical composition for the induction of apoptosis of claim 5, which is useful for the prevention or treatment of skin cancer.

8. A cosmetic composition for the induction of apoptosis containing phytosphingosine derivatives as effective components.

9. The cosmetic composition for the induction of apoptosis of claim 8, wherein said phytosphingosine derivatives are one or more compounds selected from a group comprised of phytosphingosine, phytosphingosine-HCl, C6-phytosphingosine, CLA-phytosphingosine, tetraacetyl phytosphingosine, and N-acetyl phytosphingosine.

10. The cosmetic composition for the induction of apoptosis of claim 8, wherein vitamin D₃ or calcipotriol is contained additionally in said phytosphingosine derivatives.

11. The cosmetic composition for the induction of apoptosis of claim 8, which is useful for the prevention or treatment of various skin diseases, various tumors, various cancers, and other diseases that may be prevented or cured by the induction of the activity of apoptosis in living bodies.

12. The cosmetic composition for the induction of apoptosis of claim 12, which is useful for the prevention or treatment of abnormal skin diseases such as eczema, psoriasis, ichthyotis, and others; skin diseases such as atopic dermatitis, dermatitis, itching, microbism, acne, wound, and others; abnormal skin diseases induced by a long-time exposure to UV light and dermal aging; and skin cancer and others.

13. The cosmetic composition for the induction of apoptosis of claim 12, which is useful for the prevention or treatment of abnormal skin diseases such as psoriasis, ichthyosis, and others; and abnormal skin diseases induced by a long-time exposure to UV light and dermal aging.

14. The cosmetic composition for the induction of apoptosis of claim 12, which is useful for the treatment of skin cancer.

15. A method of prevention or treatment of various skin diseases, various tumors, various cancers, and other diseases that may be prevented or cured by the induction of the activity of apoptosis in living bodies, comprising the steps of the administration of compositions for the induction of apoptosis containing phytosphingosine derivatives as effective components and of the irradiation of UVB to a psoriatic lesion.

16. The method of prevention or treatment of various skin diseases, various tumors, various cancers, and other diseases of claim 15, wherein said phytosphingosine derivatives are one or more compounds selected from a group comprised of phytosphingosine, phytosphingosine-HCl, C6-phytosphingosine, CLA-phytosphingosine, tetraacetyl phytosphingosine, and N-acetyl phytosphingosine.

17. The method of prevention or treatment of claim 15, wherein abnormal skin diseases such as eczema, psoriasis, ichthyotis, and others; skin diseases such as atopic dermatitis, dermatitis, itching, microbism, acne, wound, and others; abnormal skin diseases induced by a long-time exposure to UV light and dermal aging; and skin cancer and others are prevented or treated.

18. The method of prevention or treatment of claim 17, wherein abnormal skin diseases such as psoriasis, ichthyosis, and others and abnormal skin diseases induced by a long-time exposure to UV light and dermal aging are prevented or treated.

19. The method of prevention or treatment of claim 17, wherein skin cancer is prevented or treated.
